(19) United States
(12) Patent Application Publication
(10) Pub. No.: US 2014/0276026 A1
(43) Pub. Date: Sep. 18, 2014

(54) DEVICES AND METHODS FOR IMAGING AND DELIVERING A FERTILIZED EGG INTO A WOMAN'S UTERUS

(71) Applicant: VOLCANO CORPORATION, San Diego, CA (US)

(72) Inventor: David Goodman, Carlsbad, CA (US)

(73) Assignee: VOLCANO CORPORATION, San Diego, CA (US)

(21) Appl. No.: 14/207,923

(22) Filed: Mar. 13, 2014

Related U.S. Application Data
(60) Provisional application No. 61/778,793, filed on Mar. 13, 2013.

(51) Int. Cl. A61B 5/00; A61B 8/00; A61B 8/12; A61B 17/435; A61B 17/00

(52) U.S. Cl. CPC A61B 5/6852; A61B 17/435; A61B 17/00234; A61B 5/4836; A61B 8/12; A61B 5/0095; A61B 8/4416
USPC 600/427; 600/407; 600/439

(57) ABSTRACT

The invention generally relates to devices and methods for imaging and delivering a fertilized egg into a woman's uterus. In certain embodiments, the invention provides devices that include a body configured to fit within a lumen of a woman's reproductive system. The body includes an opening. Devices of the invention also include a channel within the body. The channel includes a distal end that is connected to the opening. Devices of the invention also include an imaging assembly coupled to the body.

### RELATED APPLICATION

The present application claims the benefit of and priority to U.S. provisional patent application Ser. No. 61/778,793, filed Mar. 13, 2013, the content of which is incorporated by reference herein in its entirety.

### FIELD OF THE INVENTION

The invention generally relates to devices and methods for imaging and delivering a fertilized egg into a woman's uterus.

### BACKGROUND

Approximately one in seven couples have difficulty conceiving. Infertility may be due to a single cause in either partner, or a combination of factors (e.g., genetic factors, diseases, or environmental factors) that may prevent a pregnancy from occurring or continuing.

In vitro fertilization (IVF), a process in which egg cells are fertilized by sperm outside a woman's womb and then implanted into the womb, is a common procedure to assist women who have difficulty conceiving. The implantation process for IVF involves having a woman lie on a table or bed, while a doctor exposes her cervix. One or more embryos suspended in a drop of culture medium are drawn into a transfer catheter. Gently, the doctor guides the tip of the loaded catheter through the cervix and deposits the fluid containing the embryos into the uterine cavity. The procedure may be visualized using abdominal ultrasound, to ensure proper placement of the embryos in the uterine cavity. Abdominal ultrasound may be uncomfortable for the woman as it requires a lot of pressure in order to visualize the depth through all of the layers of tissue.

### SUMMARY

The invention generally relates to devices and methods that allow for real-time internal imaging of the uterine cavity during an in vitro fertilization (IVF) procedure. Aspects of the invention are accomplished by providing a device with an integrated imaging assembly. Such a device allows an operator to see within the uterine cavity and to deliver the fertilized embryos while visualizing the uterine cavity with the same device.

In certain aspects, devices of the invention include a body configured to fit within a lumen of a woman's reproductive system, the body having an opening. Within the body there is a channel. A distal end of that channel is connected to the opening. There is also an imaging assembly coupled to the body. Devices of the present invention may be used in a variety of body lumens, including but not limited to intravascular lumens of a woman's reproductive system.

The body of devices of the invention generally includes a proximal and a distal portion. The distal portion generally includes the opening. The opening may be located at a distal end of the body or may be located along a sidewall of the body. In certain embodiments, the opening is located on a sidewall in a distal portion of the body. The opening may be any size. The body may have any configuration that allows it to fit within a lumen of a vessel. Generally, the opening may include a slidable cover that is closed during insertion of the device into a vessel lumen, and opened once the catheter is properly positioned within the uterus. In certain embodiments, the device is a catheter, and the opening is located on a sidewall or distal end of the catheter.

The channel generally runs the length of the body and is coaxial with the length of the body. The channel has a distal end that is coupled to the opening. In certain embodiments, the channel may be integrally formed with the body. The channel may have any inner diameter.

The catheter body generally includes a proximal portion and a distal portion, with the distal portion having the opening. In catheter embodiments, the catheter may have many various sizes and configurations. The proximal portion and the distal portion of the catheter body typically define a channel having a longitudinal axis.

In devices and methods of the invention, an imaging assembly is coupled to the body. In certain embodiments, the imaging assembly is positioned to allow imaging of an opening in the device. Such placement of the imaging assembly greatly improves visualization during the thrombolysis procedure. Any imaging assembly may be used with devices and methods of the invention, such as opto-acoustic sensor apparatuses, intravascular ultrasound (IVUS) or optical coherence tomography (OCT).

In certain embodiments, the imaging assembly includes at least one opto-acoustic sensor. Generally, the opto-acoustic sensor will include an optical fiber having a blazed fiber Bragg grating, a light source that transmits light through the optical fiber, and a photoacoustic transducer material positioned so that it receives light diffracted by the blazed fiber Bragg grating and emits ultrasonic imaging energy. The sensor may be positioned on an internal wall of the device, opposite the opening. In certain embodiments, the at least one sensor is a plurality of sensors and the sensors are arranged in a semi-circle.

Another aspect of the invention provides methods for imaging and delivering a fertilized egg into a woman's uterus that involve providing a device that includes a body configured to fit within a lumen of a woman's reproductive system. Within the body there is a channel. A distal end of that channel is connected to the opening. There is also an imaging assembly coupled to the body. The method further involves inserting the device into a woman's uterus, and simultaneously delivering a fertilized egg into the woman's uterus while imaging within the uterus.

### BRIEF DESCRIPTION OF THE DRAWINGS

FIG. 1 shows an exemplary embodiment of a side view of a device of the invention in which the imaging assembly looks sideways.

FIG. 2A shows another view of FIG. 1, illustrating a guidewire in a channel that is distinct from the drug delivery channel.

FIG. 2B shows another embodiment of devices of the invention in which a single channel is both the drug delivery channel and a guidewire channel.

FIG. 3 shows a connector fitting that connects to devices of the invention.

FIG. 4A shows an exemplary embodiment of a side view of a device of the invention in which the imaging assembly looks sideways and is positioned to image the opening.

FIG. 4B shows an exemplary embodiment of a side view of a device of the invention in which the imaging assembly looks forward.

FIG. 5 is a schematic diagram of a conventional optical fiber.

FIG. 6 is a cross-sectional schematic diagram illustrating generally one example of a distal portion of an imaging assembly that combines an acousto-optic Fiber Bragg Grating (FBG) sensor with an photoacoustic transducer.

FIG. 7 is a schematic diagram of a Fiber Bragg Grating based sensor

FIG. 8 is a cross-sectional schematic diagram illustrating generally one example of the operation of a blazed grating FBG photoacoustic transducer.

FIG. 9 is a schematic diagram illustrating generally one technique of generating an image by rotating the blazed FBG optical-to-acoustic and acoustic-to-optical combined transducer and displaying the resultant series of radial image lines to create a radial image.

FIG. 10 is a schematic diagram that illustrates generally one such phased array example, in which the signal to/from each array transducer is combined with the signals from the other transducers to synthesize a radial image line.

FIG. 11 is a schematic diagram that illustrates generally an example of a side view of a distal portion of a device.

FIG. 12 is a schematic diagram that illustrates generally one example of a cross-sectional side view of a distal portion of a device.

### DETAILED DESCRIPTION

The invention generally relates to devices and methods for imaging and delivering a fertilized egg into a woman's uterus. FIG. 1 shows an exemplary embodiment of a side view of a device of the invention. Devices of the invention include a body 1000 configured to fit within a lumen of a woman's reproductive system, the body having an opening 1001. Within the body 1000 there is a channel 1002. A distal end of that channel is connected to the opening 1000. There is also an imaging assembly 1003 coupled to the body 1000. FIG. 1 shows that the imaging assembly 1003 emits a signal that produces an image of an inside of a vessel. That image may overlap with the position of the opening 1001 in the body 1000. In this manner, an operator can see within the uterine cavity and can a fertilized embryo while visualizing within the cavity with the same device. In this embodiment, the imaging assembly 1003 is placed distal to the opening 1001. This is only an exemplary configuration of devices of the invention and other configurations will be discussed in greater detail below.

The body 1000 generally includes a proximal and a distal portion. The distal portion generally includes the opening 1001. The opening 1001 may be located at a distal end of the body 1000 or may be located along a sidewall of the body 1000. FIG. 1 shows the opening 1001 located on a sidewall of the body 1000. The body 1000 may have any configuration that allows it to fit within a lumen of a vessel. In certain embodiments, the opening 1001 may include a slidable cover (not shown) that is closed during insertion of the device into the lumen of the woman's reproductive system, and opened once the opening 1001 is properly positioned near an obstruction.

In certain embodiments, the device is a catheter and the body is a catheter body. The catheter and catheter body are configured for introduction into a lumen female's reproductive system. intraluminal introduction to the target body lumen. The dimensions and other physical characteristics of the catheter bodies will vary significantly depending on the body lumen that is to be accessed. In the exemplary case, the catheter will be 23 cm long and the tip is 2.8 French size. The catheter will is configured and has a hardness of standard IVF catheters, such as those described in Setta et al. (Human Reproduction, 20(11):3114-3121, 2005), and those commercial available from Smiths Medical.

Catheters of the invention may be used with a guidewire or without a guidewire. FIG. 1 shows an exemplary embodiment that includes an exemplary guidewire channel 1004. In certain embodiments, catheters of the invention will not have a guidewire channel. In the exemplary embodiment shown in FIG. 1, the guidewire channel 1004 is separate from the embryo delivery channel 1002. Another view of this embodiment in shown in FIG. 2A, which shows the guidewire 1008 in a distinct guidewire channel 1004. That guidewire channel 1004 is separate from embryo delivery channel 1002. In other embodiments, the device is a single channel device. FIG. 2B shows such a device.

Additionally, the configuration of the guidewire channel 1004 being situated below the drug delivery channel 1002 in FIG. 1 is only exemplary. Any configuration of the two channels is within the scope of the invention. For example, the guidewire channel 1004 could be situated on top of the drug delivery channel or the guidewire channel 1004 could be side-by-side the drug delivery channel 1002. In other cases, it may be possible to provide a fixed or integral coil tip or guidewire tip on the distal portion of the catheter or even dispense with the guidewire entirely. For convenience of illustration, guidewires will not be shown in all embodiments, but it should be appreciated that they can be incorporated into any of these embodiments.

Catheter bodies will typically be composed of an organic polymer that is fabricated by conventional extrusion techniques. Suitable polymers include polyvinylchloride, polyurethanes, polyesters, polytetrafluoroethylenes (PTFE), silicone rubbers, natural rubbers, and the like. Optionally, the catheter body may be reinforced with braid, helical wires, coils, axial filaments, or the like, in order to increase rotational strength, column strength, toughness, pushability, and the like. Suitable catheter bodies may be formed by extrusion, with one or more channels being provided when desired. The catheter diameter can be modified by heat expansion and shrinkage using conventional techniques. The resulting catheters will thus be suitable for introduction to the vascular system, often the coronary arteries, by conventional techniques.

The distal portion of the catheters of the present invention may have a wide variety of forms and structures. In many embodiments, a distal portion of the catheter is more rigid than a proximal portion, but in other embodiments the distal portion may be equally as flexible as the proximal portion. One aspect of the present invention provides catheters having a distal portion with a reduced rigid length. The reduced rigid length can allow the catheters to access and treat tortuous vessels and small diameter body lumens. In most embodiments a rigid distal portion or housing of the catheter body will have a diameter that generally matches the proximal portion of the catheter body, however, in other embodiments, the distal portion may be larger or smaller than the flexible portion of the catheter.

A rigid distal portion of a catheter body can be formed from materials that are rigid or which have very low flexibilities, such as metals, hard plastics, composite materials, NiTi, steel with a coating such as titanium nitride, tantalum, ME-92 (antibacterial coating material), diamonds, or the like. Most usually, the distal end of the catheter body will be formed from stainless steel or platinum/iridium. The length of the rigid distal portion may vary widely, typically being in the range from 5 mm to 35 mm, more usually from 10 mm to 25 mm, and preferably between 6 mm and 8 mm. In contrast, conventional catheters typically have rigid lengths of approximately 16 mm. The opening 1001 of the present invention will typically have a length of approximately 2 mm. In other embodiments, however, the opening can be larger or smaller.

The catheter may include a flexible atraumatic distal tip coupled to the rigid distal portion of the catheter. For example, an integrated distal tip can increase the safety of the catheter by eliminating the joint between the distal tip and the catheter body. The integral tip can provide a smoother inner diameter for ease of tissue movement. During manufacturing, the transition from the housing to the flexible distal tip can be finished with a polymer laminate over the material housing. No weld, crimp, or screw joint is usually required.

The atraumatic distal tip permits advancing the catheter distally through a body lumen while reducing any damage caused to the body lumen by the catheter. Typically, the distal tip will have a guidewire channel to permit the catheter to be guided to the target over a guidewire. In some exemplary configurations, the atraumatic distal tip includes a coil. In some configurations the distal tip has a rounded, blunt distal end. The catheter body can be tubular and have a forward-facing circular aperture which communicates with the atraumatic tip.

The body 1000 includes an embryo delivery channel 1002 extending through the body 1000. A distal end of the channel 1002 is coupled to the opening 1001, and a proximal end of the channel is configured for introduction of the fertilized embryos. In certain embodiments, the channel 1002 is connected to a fluid pump via a connector fitting 1005. Connector fitting 1005 is attached at the proximal end of the body 1000. Connector fitting 1005 provides a functional access port at the proximal end of devices of the invention. Connector fitting 1005 is attached to the body 1000 and has a central passageway 1006 in communication with the channel 1002 to allow passage of various fluids, such as saline or other buffered fluids. Connector fitting 1005 further includes an adaptor 1007 in fluid communication with channel 1002 and adapted for connection to a fluid pump (not shown) to deliver fluids to channel 1002.

The adapter 1007 is configured to sealably mate to an outlet of a drug delivery device. Such sealable mating can be by any method known in the art. For example, the adaptor 1007 can be a female connector piece that sealably mates with a male connector of a drug delivery device. Alternatively, the adaptor 1007 can be a male connector piece that sealably mates with a female connector of a drug delivery device. In certain embodiments, the adapter 1007 includes a gasket, such as an elastomeric gasket that allows for sealable mating to the drug delivery devices. Elastomeric gaskets are described for example in Leblanc et al. (U.S. patent publication number 2012/0244043), the content of which is incorporated by reference herein in its entirety.

Devices of the invention also include an imaging assembly 1003 coupled to the body 1000. The imaging assembly may be placed distal to the opening 1001 (as shown in FIG. 1) or positioned elsewhere, such as proximal to the opening. The imaging assembly 1003 can be angled to image perpendicular to the opening 1001 (as shown in FIG. 1) or could be angled to image just forward or behind the opening depending on the position of the imaging assembly 1003. In certain embodiments, the imaging assembly 1003 is positioned to imaging the opening 1001 in the device (FIG. 4A). In embodiments in which the opening 1001 is at a distal end of the body 1000, the imaging assembly may be placed next to the opening to image forward (FIG. 4B).

Any imaging assembly may be used with devices and methods of the invention, such as optical-acoustic imaging apparatus, intravascular ultrasound (IVUS), forward-looking intravascular ultrasound (FLIVUS) or optical coherence tomography (OCT). In certain embodiments, the imaging assembly is an optical-acoustic imaging apparatus. Exemplary optical-acoustic imaging sensors are shown for example in, U.S. Pat. No. 7,245,789; U.S. Pat. No. 7,447,388; U.S. Pat. No. 7,660,492; U.S. Pat. No. 8,059,923; US 2012/0108943; and US 2010/0087732, the content of each of which is incorporated by reference herein in its entirety. Additional optical-acoustic sensors are shown for example in U.S. Pat. No. 6,659,957; U.S. Pat. No. 7,527,594; and US 2008/0119739, the content of each of which is incorporated by reference herein in its entirety.

An exemplary optical-acoustic imaging apparatus includes a photoacoustic transducer and a blazed Fiber Bragg grating. Optical energy of a specific wavelength travels down a fiber core of optical fiber and is reflected out of the optical fiber by the blazed grating. The outwardly reflected optical energy impinges on the photoacoustic material. The photoacoustic material then generates a responsive acoustic impulse that radiates away from the photoacoustic material toward nearby biological or other material to be imaged. Acoustic energy of a specific frequency is generated by optically irradiating the photoacoustic material at a pulse rate equal to the desired acoustic frequency.

The optical-acoustic imaging apparatus utilizes at least one and generally more than one optical fiber, for example but not limited to a glass fiber at least partly composed of silicon dioxide. The basic structure of a generic optical fiber is illustrated in FIG. 5, which fiber generally consists of layered glass cylinders. There is a central cylinder called the core 1. Surrounding this is a cylindrical shell of glass, possibly multilayered, called the cladding 2. This cylinder is surrounded by some form of protective jacket 3, usually of plastic (such as acrylate). For protection from the environment and more mechanical strength than jackets alone provide, fibers are commonly incorporated into cables. Typical cables have a polyethylene sheath 4 that encases the fibers within a strength member 5 such as steel or Kevlar strands.

FIG. 6 is a cross-sectional schematic diagram illustrating generally one example of a distal portion of an imaging assembly that combines an acousto-optic Fiber Bragg Grating (FBG) sensor 100 with an photoacoustic transducer 325. The optical fiber includes a blazed Fiber Bragg grating. Fiber Bragg Gratings form an integral part of the optical fiber structure and can be written intracore during manufacture or after manufacture. As illustrated in FIG. 7, when illuminated by a broadband light laser 7, a uniform pitch Fiber Bragg Grating element 8 will reflect back a narrowband component centered about the Bragg wavelength λ given by λ=2nλ, where n is the index of the core of the fiber and λ represents the grating period. Using a tunable laser 7 and different grating periods (each period is approximately 0.5 μm) situated in different positions on the fiber, it is possible to make independent measurement in each of the grating positions.

Referring back to FIG. 6, unlike an unblazed Bragg grating, which typically includes impressed index changes that are substantially perpendicular to the longitudinal axis of the fiber core 115 of the optical fiber 105, the blazed Bragg grating 330 includes obliquely impressed index changes that are at a nonperpendicular angle to the longitudinal axis of the optical fiber 105. As mentioned above, a standard unblazed FBG partially or substantially fully reflects optical energy of a specific wavelength traveling down the axis of the fiber core 115 of optical fiber 105 back up the same axis. Blazed FBG 330 reflects this optical energy away from the longitudinal axis of the optical fiber 105. For a particular combination of blaze angle and optical wavelength, the optical energy will leave blazed FBG 330 substantially normal (i.e., perpendicular) to the longitudinal axis of the optical fiber 105. In the illustrative example of FIG. 22, an optically absorptive photoacoustic material 335 (also referred to as a “photoacoustic” material) is placed on the surface of optical fiber 105. The optically absorptive photoacoustic material 335 is positioned, with respect to the blazed grating 330, so as to receive the optical energy leaving the blazed grating. The received optical energy is converted in the optically absorptive material 335 to heat that expands the optically absorptive photoacoustic material 335. The optically absorptive photoacoustic material 335 is selected to expand and contract quickly enough to create and transmit an ultrasound or other acoustic wave that is used for acoustic imaging of the region of interest.

FIG. 8 is a cross-sectional schematic diagram illustrating generally one example of the operation of photoacoustic transducer 325 using a blazed Bragg grating 330. Optical energy of a specific wavelength, λ1, travels down the fiber core 115 of optical fiber 105 and is reflected out of the optical fiber 105 by blazed grating 330. The outwardly reflected optical energy impinges on the photoacoustic material 335. The photoacoustic material 335 then generates a responsive acoustic impulse that radiates away from the photoacoustic material 335 toward nearby biological or other material to be imaged. Acoustic energy of a specific frequency is generated by optically irradiating the photoacoustic material 335 at a pulse rate equal to the desired acoustic frequency.

In another example, the photoacoustic material 335 has a thickness 340 (in the direction in which optical energy is received from blazed Bragg grating 330) that is selected to increase the efficiency of emission of acoustic energy. In one example, thickness 340 is selected to be about ¼ the acoustic wavelength of the material at the desired acoustic transmission/reception frequency. This improves the generation of acoustic energy by the photoacoustic material.

In yet a further example, the photoacoustic material is of a thickness 300 that is about ¼ the acoustic wavelength of the material at the desired acoustic transmission/reception frequency, and the corresponding glass-based optical fiber sensing region resonant thickness 300 is about ½ the acoustic wavelength of that material at the desired acoustic transmission/reception frequency. This further improves the generation of acoustic energy by the photoacoustic material and reception of the acoustic energy by the optical fiber sensing region.

In one example of operation, light reflected from the blazed grating excites the photoacoustic material in such a way that the optical energy is efficiently converted to substantially the same acoustic frequency for which the FBG sensor is designed. The blazed FBG and photoacoustic material, in conjunction with the aforementioned FBG sensor, provide both a transmit transducer and a receive sensor, which are harmonized to create an efficient unified optical-to-acoustic-to-optical transmit/receive device. In one example, the optical wavelength for sensing is different from that used for transmission. In a further example, the optical transmit/receive frequencies are sufficiently different that the reception is not adversely affected by the transmission, and vice-versa.

FIG. 9 is a schematic diagram illustrating generally one technique of generating an image of biological material and a vessel wall 600 through an opening in a device. The technique involves rotating the blazed FBG optical-to-acoustic and acoustic-to-optical combined transducer 500 and displaying the resultant series of radial image lines to create a radial image. In another example, phased array images are created using a substantially stationary (i.e., non-rotating) set of multiple FBG sensors, such as FBG sensors 500A-J. FIG. 10 is a schematic diagram that illustrates generally one such phased array example, in which the signal to/from each array transducer 500A-J is combined with the signals from one or more other transducers 500A-J to synthesize a radial image line. In this example, other image lines are similarly synthesized from the array signals, such as by using specific changes in the signal processing used to combine these signals.

FIG. 11 is a schematic diagram that illustrates generally an example of a side view of a distal portion 800 of an elongate device 805. In this example, the distal portion 800 of the device 805 includes one or more openings 810A, 810B, . . . , 810N located slightly or considerably proximal to a distal tip 815 of the device 805. Each opening 810 includes one or more optical-to-acoustic transducers 325 and a corresponding one or more separate or integrated acoustic-to-optical FBG sensors 100. In one example, each opening 810 includes an array of blazed FBG optical-to-acoustic and acoustic-to-optical combined transducers 500 (such as illustrated in FIG. 10) located slightly proximal to distal tip 815 of device 805 having mechanical properties that allow the device 805 to be guided through a vascular or other lumen.

FIG. 12 is a schematic diagram that illustrates generally one example of a cross-sectional side view of a distal portion 900 of another device 905. In this example, optical fibers 925 are distributed around a bottom portion of device 905. In this example, the optical fibers 925 are at least partially embedded in a polymer matrix or other binder material that bonds the optical fibers 925 to the device 905. The binder material may also contribute to the torsion response of the resulting device 905. In one example, the optical fibers 925 and binder material is overcoated with a polymer or other coating 930, such as for providing abrasion resistance, optical fiber protection, and/or friction control.

In one example, before the acoustic transducer(s) is fabricated, the device 905 is assembled, such as by binding the optical fibers 925 to the device 905, and optionally coating the device 905. The opto-acoustic transducer(s) are then integrated into the imaging assembly, such as by grinding one or more grooves in the device wall at locations of the opto-acoustic transducer window 810. In a further example, the depth of these groove(s) in the optical fiber(s) 925 defines the resonant structure(s) of the opto-acoustic transducer(s).

After the opto-acoustic transducer windows 810 have been defined, the FBGs added to one or more portions of the optical fiber 925 within such windows 810. In one example, the FBGs are created using an optical process in which the portion of the optical fiber 925 is exposed to a carefully controlled pattern of UV radiation that defines the Bragg gratings. Then, a photoacoustic material is deposited or otherwise added in the transducer windows 810 over respective Bragg gratings. One example of a suitable photoacoustic material is pigmented polydimethylsiloxane (PDMS), such as a mixture of PDMS, carbon black, and toluene.

An opto-electronics module may include one or more lasers and fiber optic elements. In one example, such as where different transmit and receive wavelengths are used, a first laser is used for providing light to the imaging assembly 905 for the transmitted ultrasound, and a separate second laser is used for providing light to the imaging assembly 905 for being modulated by the received ultrasound. In this example, a fiber optic multiplexer couples each channel (associated with a particular one of the optical fibers 925) to the transmit and receive lasers and associated optics. This reduces system complexity and costs.

In one example, the sharing of transmit and receive components by multiple guidewire channels is possible at least in part because the acoustic image is acquired over a relatively short distance (e.g., millimeters). The speed of ultrasound in a human or animal body is slow enough to allow for a large number of transmit/receive cycles to be performed during the time period of one image frame. For example, at an image depth (range) of about 2 cm, it will take ultrasonic energy approximately 26 microseconds to travel from the sensor to the range limit, and back. In one such example, therefore, an about 30 microseconds transmit/receive (T/R) cycle is used. In the approximately 30 milliseconds allotted to a single image frame, up to 1,000 T/R cycles can be carried out. In one example, such a large number of T/R cycles per frame allows the system to operate as a phased array even though each sensor is accessed in sequence. Such sequential access of the photoacoustic sensors in the guidewire permits (but does not require) the use of one set of T/R opto-electronics in conjunction with a sequentially operated optical multiplexer. In one example, instead of presenting one 2-D slice of the anatomy, the system is operated to provide a 3-D visual image that permits the viewing of a desired volume of the patient's anatomy or other imaging region of interest. This allows the physician to quickly see the detailed spatial arrangement of structures with respect to other anatomy.

In one example, in which the imaging assembly 905 includes 30 sequentially-accessed optical fibers having up to 10 photoacoustic transducer windows per optical fiber, 30×10=300 T/R cycles are used to collect the image information from all the openings for one image frame. This is well within the allotted 1,000 such cycles for a range of 2 cm, as discussed above. Thus, such an embodiment allows substantially simultaneous images to be obtained from all 10 openings at of each optical fiber at video rates (e.g., at about 30 frames per second for each transducer window). This allows real-time volumetric data acquisition, which offers a distinct advantage over other imaging techniques.

In another embodiment, the imaging assembly uses intravascular ultrasound (IVUS). IVUS imaging assemblies and processing of IVUS data are described for example in Yock, U.S. Pat. Nos. 4,794,931, 5,000,185, and 5,313,949; Sieben et al., U.S. Pat. Nos. 5,243,988, and 5,353,798; Crowley et al., U.S. Pat. No. 4,951,677; Pomeranz, U.S. Pat. No. 5,095,911, Griffith et al., U.S. Pat. No. 4,841,977, Maroney et al., U.S. Pat. No. 5,373,849, Born et al., U.S. Pat. No. 5,176,141, Lancee et al., U.S. Pat. No. 5,240,003, Lancee et al., U.S. Pat. No. 5,375,602, Gardineer et at., U.S. Pat. No. 5,373,845, Seward et al., Mayo Clinic Proceedings 71(7):629-635 (1996), Packer et al., Cardiostim Conference 833 (1994), “Ultrasound Cardioscopy,” Eur. J. C. P. E. 4(2):193 (June 1994), Eberle et al., U.S. Pat. No. 5,453,575, Eberle et al., U.S. Pat. No. 5,368,037, Eberle et at., U.S. Pat. No. 5,183,048, Eberle et al., U.S. Pat. No. 5,167,233, Eberle et at., U.S. Pat. No. 4,917,097, Eberle et at., U.S. Pat. No. 5,135,486, and other references well known in the art relating to intraluminal ultrasound devices and modalities.

In another embodiment, the imaging assembly uses optical coherence tomography (OCT). OCT is a medical imaging methodology using a miniaturized near infrared light-emitting probe. As an optical signal acquisition and processing method, it captures micrometer-resolution, three-dimensional images from within optical scattering media (e.g., biological tissue). Recently it has also begun to be used in interventional cardiology to help diagnose coronary artery disease. OCT allows the application of interferometric technology to see from inside, for example, blood vessels, visualizing the endothelium (inner wall) of blood vessels in living individuals.

OCT systems and methods are generally described in Castella et al., U.S. Pat. No. 8,108,030, Milner et al., U.S. Patent Application Publication No. 2011/0152771, Condit et al., U.S. Patent Application Publication No. 2010/0220334, Castella et al., U.S. Patent Application Publication No. 2009/0043191, Milner et al., U.S. Patent Application Publication No. 2008/0291463, and Kemp, N., U.S. Patent Application Publication No. 2008/0180683, the content of each of which is incorporated by reference in its entirety.

Some exemplary methods of the present invention will now be described. One method of the present invention includes delivering a device to a woman's uterine cavity. Once within the cavity, a slidable cover on the opening is retracted and the imaging assembly is activated. This allows the images of the uterine cavity while the fertilized eggs are implanted.

The device can be percutaneously advanced through a guide catheter or sheath and over a conventional or imaging guidewire using conventional interventional techniques. The device can be advanced over the guidewire and out of the guide catheter to the diseased area. If there is a cover, the opening will typically be closed. Although, a cover is not required. The device will typically have at least one hinge or pivot connection to allow pivoting about one or more axes of rotation to enhance the delivery of the catheter into the tortuous anatomy without dislodging the guide catheter or other sheath. The device can be positioned within the uterine cavity.

Once positioned, fertilized embryos are delivered to within the cavity via the channel. The imaging data is used to guide the operator during the implantation process. When it is determined that the embryos have been implanted, the catheter can be removed from the body lumen.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A device for imaging and delivering a fertilized egg into a woman's uterus, the device comprising:
a body configured to fit within a lumen of a woman's reproductive system, the body comprising an opening;
a channel within the body comprising a distal end that is connected to the opening; and
an imaging assembly coupled to the body.

2. The device according to claim 1, wherein the device is a catheter.

3. The device according to claim 2, wherein the opening is on a side of the catheter.

4. The device according to claim 2, wherein the opening is at a distal end of the catheter.

5. The device according to claim 1, wherein the imaging assembly is positioned to image the opening.

6. The device according to claim 5, wherein the imaging assembly is selected from the group consisting of: an ultrasound assembly and an optical coherence tomography assembly.

7. The device according to claim 6, wherein the ultrasound assembly comprises at least one opto-acoustic sensor.

8. The device according to claim 7, wherein the at least one sensor is placed on an internal wall of the catheter, opposite the opening.

9. The device according to claim 8, wherein the at least one sensor is a plurality of sensors and the sensors are arranged in a semi-circle.

10. The device according to claim 7, wherein the at least one sensor is embedded within an internal wall of the catheter, opposite the opening.

11. The device according to claim 10, wherein the at least one sensor is a plurality of sensors and the sensors are arranged in a semi-circle.

12. The device according to claim 7, wherein the opto-acoustic sensor comprises:
an optical fiber comprising a blazed fiber Bragg grating;
a light source that transmits light through the optical fiber; and
a photoacoustic transducer material positioned so that it receives light diffracted by the blazed fiber Bragg grating and emits ultrasonic imaging energy.

13. The device according to claim 1, wherein the imaging assembly is configured to image forward of the device.

14. A method for imaging and delivering a fertilized egg into a woman's uterus, the method comprising:
providing a device comprising: a body configured to fit within a lumen of a woman's reproductive system, the body comprising an opening; a channel within the body comprising a distal end that is connected to the opening; and an imaging assembly coupled to the body;
inserting the device into a woman's uterus;
simultaneously delivering a fertilized egg into the woman's uterus while imaging within the uterus.

15. The method according to claim 14, wherein the device is a catheter.

16. The method according to claim 15, wherein the opening is on a side of the catheter.

17. The method according to claim 16, wherein the opening is at a distal end of the catheter.

18. The device according to claim 14, wherein the imaging assembly is positioned to image the opening.

19. The method according to claim 18, wherein the imaging assembly is selected from the group consisting of: an ultrasound assembly and an optical coherence tomography assembly.

20. The method according to claim 19, wherein the ultrasound assembly comprises at least one opto-acoustic sensor.

21. The method according to claim 20, wherein the at least one sensor is placed on an internal wall of the catheter, opposite the opening.

22. The method according to claim 21, wherein the at least one sensor is a plurality of sensors and the sensors are arranged in a semi-circle.

23. The method according to claim 20, wherein the at least one sensor is embedded within an internal wall of the catheter, opposite the opening.

24. The method according to claim 23, wherein the at least one sensor is a plurality of sensors and the sensors are arranged in a semi-circle.

25. The method according to claim 20, wherein the opto-acoustic sensor comprises:
an optical fiber comprising a blazed fiber Bragg grating;
a light source that transmits light through the optical fiber; and
a photoacoustic transducer material positioned so that it receives light diffracted by the blazed fiber Bragg grating and emits ultrasonic imaging energy.

26. The method according to claim 14, wherein the imaging assembly is configured to image forward of the device.
